(19) United States
(12) Patent Application Publication
(10) Pub. No.: US 2023/0287048 A1
(43) Pub. Date: Sep. 14, 2023

(54) COMPOSITION AND KIT FOR REMOVING LIPOPOLYSACCHARIDE

(71) Applicant: HLB SCIENCE INC., Seoul (KR)

(72) Inventors: In Duk JUNG, Chungcheongbuk-do (KR); Yong Joo KIM, Gyeonggi-do (KR); Mi Suk LEE, Seoul (KR); Hee Jo PARK, Seoul (KR)

(21) Appl. No.: 17/906,763

(22) PCT Filed: Mar. 18, 2021
(86) PCT No.: PCT/KR2021/003386

§ 371 Date: Sep. 20, 2022

(30) Foreign Application Priority Data
Mar. 20, 2020 (KR) 10-2020-0034586
Mar. 17, 2021 (KR) 10-2021-0034949

(51) Int. Cl. C07K 7/08; C07K 1/14; C07K 1/04

(52) U.S. Cl. CPC C07K 7/08; C07K 1/14; C07K 1/042

(57) ABSTRACT

The present invention relates to a composition and a kit for removing lipopolysaccharide (LPS), comprising a polypeptide having binding ability to lipopolysaccharide or a salt substitute thereof as an active ingredient, and a method for removing lipopolysaccharide. The polypeptide or a salt substitute thereof according to the present invention has very excellent binding ability with lipopolysaccharide, and the efficiency of removing lipopolysaccharide during a purification process in a protein production process using E. coli is high, such that the lipopolysaccharide removal efficiency can be maximized.

### CROSS-REFERENCE TO RELATED APPLICATIONS

This application is a national phase application filed under 35 U.S.C. § 371 claiming benefit to International Patent Application No. PCT/KR2021/003386, filed Mar. 18, 2021, which claims the benefit of priority from KR Patent Application No. 10-2020-0034586, filed Mar. 20, 2020, and KR Patent Application No. 10 2021-0034949, filed Mar. 17, 2021, each of which is hereby incorporated by reference in its entirety herein.

### REFERENCE TO A “SEQUENCE LISTING”, A TABLE, OR A COMPUTER PROGRAM LISTING APPENDIX SUBMITTED AS AN ASCII TEXT FILE

The present application hereby incorporates by reference the entire contents of the text file named “2016-0134-00US_SequenceListing.txt” in ASCII format, which was created on Sep. 6, 2022, and is 4,069 bytes in size.

### TECHNICAL FIELD

The present invention relates to a composition and kit for removing lipopolysaccharide (LPS); and a method of removing lipopolysaccharide.

This application claims priority to and the benefits of Korean Patent Application No. 10-2020-0034586, filed on Mar. 20, 2020, and Korean Patent Application No. 10-2021-0034949, filed on Mar. 17, 2021, the disclosures of which are incorporated herein by reference in their entirety.

### BACKGROUND ART

Lipopolysaccharide (LPS) consists of lipid A, a core polysaccharide, and an 0-specific polysaccharide side chain. LPS is heat-stable and exhibits strong antigenicity. LPS forms multiple complexes with a basic unit of 10 kDa, and its size varies up to 10,000 kDa, and is an amphoteric compound because of its chemical structure.

LPS causes an inflammatory response in animals, and in most cases, is present in the outer membrane of the cell wall of gram-negative bacteria such as Salmonella and E. coli. The toxic part of LPS, which is the cause of such an inflammatory reaction, is a lipid part, called endotoxin. When such endotoxin enters the human body, fever, a change in white blood cell count, blood vessel coagulation, tumor necrosis, hypertension and shock occur, and endotoxin causes the production of various cytokines such as IL-1, IL-6, IL-8 and TNF, the activation of an immune system complement, and the activation of blood clotting and acts as a mitogen to increase polyclonal differentiation of B cells, proliferation of B cells, and production of IgM and IgG antibodies.

Endotoxin refers to the LPS family that forms the outer cell membrane of gram-negative bacteria together with proteins and phospholipids. Endotoxin is generated only in this bacterium group and plays an important role in the organization, stability and barrier function of the outer cell membrane. Many bacteriophages use endotoxins or general LPS as a specific method for finding their host bacteria.

Endotoxin is present in two molecules and may be found in almost all water-soluble solutions without any special precautions. In humans and animals, endotoxin causes sepsis, leading to a powerful erroneous reaction of the immune system. Therefore, for example, in the case of producing a pharmaprotein, contamination with endotoxin must be precisely detected and then completely removed. Endotoxin causes problems with a genetically-treated drug, gene therapy or a related material, which is injected into a human or animal (e.g., livestock treatment or animal testing). However, inhibition of or reduction in injection efficiency by endotoxin can be observed in pharmaceutical and research applications such as an experiment for injection of mammalian cells.

In order to use a protein within the scope of clinical research, the European and US Pharmacopoeias require that the protein should be lower than a specific threshold for endotoxin levels. If the drug or protein contained as above has an excessively high endotoxin level, it can lead to the death of a test subject. The above erroneous immune defense causes damage to a patient due to overreaction. It causes tissue infection, a decrease in blood pressure, a rapid heartbeat, thrombosis, and shock. Even long-term exposure to endotoxin in picograms may cause chronic side effects such as immunodeficiency and sepsis. Within the framework of material preparation, in particular, in the process according to “Good Manufacturing Practice (GMP),” there is an attempt to remove as much endotoxin as possible. However, the removal of endotoxin from proteins, etc., in particular, has a big problem that the removal of endotoxin is actually hindered due to the protein's charge state or its intrinsic property such as hydrophobicity, or many products are lost during the removal process.

Meanwhile, in the mass production of proteins using E. coli, it is necessary to remove endotoxin as described above, and in a purification process of such proteins, a method using chromatography to remove endotoxin (Korean Patent Publication No. 2015-0118120) was used.

However, this method had many problems in purifying a large amount of protein and also had an economical problem of an increase in price in product production.

### DISCLOSURE

### Technical Problem

To solve the above-described problems, the present invention is directed to providing a composition and kit for removing lipopolysaccharide (LPS), which comprises a polypeptide or salt substitute thereof, having binding ability to lipopolysaccharide, as an active ingredient; and a method of removing lipopolysaccharide.

However, technical problems to be solved in the present invention are not limited to the above-described problems, and other problems which are not described herein will be fully understood by those of ordinary skill in the art from the following descriptions.

### Technical Solution

To achieve the object of the present invention, the present invention provides a composition and kit for removing lipopolysaccharide (LPS), which comprises a polypeptide represented by the following sequence general formula or a salt substitute thereof as an active ingredient.

In addition, the present invention provides a use of a polypeptide represented by the following sequence general formula or a salt substitute thereof for removing lipopolysaccharide (LPS):

Ln-X1-L-X2-V-X3-X4-X5-R-X6-L-X7 [General Formula]
in the above general formula,
n is 0 or 1;
L is leucine;
V is valine;
R is arginine;
X1 is lysine (K) or arginine (R);
X2 is glycine (G) or arginine (R);
X3 is glutamic acid (E) or lysine (K);
X4 is alanine (A) or leucine (L);
X5 is lysine (K), arginine (R) or leucine (L);
X6 is tyrosine (Y), alanine (A), tryptophan (W), lysine (K), or aspartic acid (D); and
X7 is aspartic acid (D) or arginine (R),
wherein, however, a polypeptide represented by the sequence of K-L-G-V-E-A-K-R-Y-L-D in the above general formula is excluded.

In one embodiment of the present invention, the polypeptide may be any one of 9 types of the polypeptides consisting of a) to i) as follows, but the present invention is not limited thereto:
a) a polypeptide in which, in the above general formula,
n is 0;
X1 is lysine (K);
X2 is glycine (G);
X3 is glutamic acid (E);
X4 is alanine (A);
X5 is lysine (K);
X6 is tyrosine (Y); and
X7 is arginine (R),
b) a polypeptide in which, in the above general formula,
n is 1;
X1 is arginine (R);
X2 is glycine (G);
X3 is glutamic acid (E);
X4 is alanine (A);
X5 is lysine (K);
X6 is tyrosine (Y); and
X7 is arginine (R),
c) a polypeptide in which, in the above general formula,
n is 1;
X1 is arginine (R);
X2 is glycine (G);
X3 is glutamic acid (E);
X4 is leucine (L);
X5 is lysine (K);
X6 is tyrosine (Y); and
X7 is arginine (R),
d) a polypeptide in which, in the above general formula,
n is 0;
X1 is lysine (K);
X2 is glycine (G);
X3 is glutamic acid (E);
X4 is alanine (A);
X5 is leucine (L);
X6 is tyrosine (Y); and
X7 is aspartic acid (D),
e) a polypeptide in which, in the above general formula,
n is 0;
X1 is arginine (R);
X2 is arginine (R);
X3 is lysine (K);
X4 is leucine (L);
X5 is arginine (R);
X6 is tyrosine (Y); and
X7 is arginine (R),
f) a polypeptide in which, in the above general formula,
n is 0;
X1 is lysine (K);
X2 is arginine (R);
X3 is lysine (K);
X4 is leucine (L);
X5 is arginine (R);
X6 is tyrosine (Y); and
X7 is arginine (R),
g) a polypeptide in which, in the above general formula,
n is 0;
X1 is lysine (K);
X2 is arginine (R);
X3 is lysine (K);
X4 is leucine (L);
X5 is arginine (R);
X6 is alanine (A); and
X7 is arginine (R),
h) a polypeptide in which, in the above general formula,
n is 0;
X1 is lysine (K);
X2 is arginine (R);
X3 is lysine (K);
X4 is leucine (L);
X5 is arginine (R);
X6 is tryptophan (W); and
X7 is arginine (R), and
i) a polypeptide in which, in the above general formula,
n is 0;
X1 is lysine (K);
X2 is arginine (R);
X3 is lysine (K);
X4 is leucine (L);
X5 is arginine (R);
X6 is lysine (K); and
X7 is arginine (R).

In one embodiment of the present invention, the polypeptide may be a peptidomimetic comprising an L-type polypeptide, a D-type polypeptide or a peptoid, or non-natural amino acids, but the present invention is not limited thereto.

In one embodiment of the present invention, an end of the polypeptide may be subjected to alkylation, PEGylation, or amidation, but the present invention is not limited thereto.

In one embodiment of the present invention, an amine group (NH₂) may be added to the C-terminus of the polypeptide, but the present invention is not limited thereto.

In one embodiment of the present invention, a salt substitute of the polypeptide may be an acetate salt substitute, for example, an acetate salt substitute of trifluoroacetic acid (TFA) of the polypeptide, but the present invention is not limited thereto.

In one embodiment of the present invention, the polypeptide or salt substitute thereof may be conjugated to a substrate, but the present invention is not limited thereto.

In one embodiment of the present invention, the substrate may be an insoluble or solid substrate, for example, an agarose bead, for example, sepharose, for example, a cyanogen bromide (CNBr)— or N-hydroxysuccinimide (NHS)-binding substrate, but the present invention is not limited thereto.

In one embodiment of the present invention, the polypeptide or salt substitute thereof; and/or the composition and/or kit comprising the polypeptide or salt substitute thereof as an active ingredient can be reused multiple times, but the present invention is not limited thereto.

In one embodiment of the present invention, the polypeptide or salt substitute thereof; and/or the composition and/or kit comprising the polypeptide or salt substitute thereof as an active ingredient may have storage stability, for example, long-term storage stability, but the present invention is not limited thereto.

In one embodiment of the present invention, the kit comprising the polypeptide or salt substitute thereof as an active ingredient may be a spin-down type or column type, but the present invention is not limited thereto.

In addition, the present invention provides a method of removing lipopolysaccharide (LPS) from a sample, which comprises the following steps:
(S1) bringing a sample into contact with a polypeptide represented by the above-described sequence general formula, or a salt substitute thereof; and
(S2) isolating the combination of the polypeptide represented by the sequence general formula or a salt substitute thereof and lipopolysaccharide from the sample.

In one embodiment of the present invention, the sample comprises a recombinant protein and lipopolysaccharide, and for example, the sample may be a culture, lysate or extract of transformed gram-negative bacteria, which comprises a recombinant protein. For example, the gram-negative bacterial may be E. coli, but the present invention is not limited thereto.

In one embodiment of the present invention, the lipopolysaccharide may comprise endotoxin, but the present invention is not limited thereto.

In one embodiment of the present invention, (S2) may be a step of isolating a combination of the polypeptide or a salt substitute thereof and the lipopolysaccharide through centrifugation of the contact product between the polypeptide or salt substitute thereof and the sample in (S1); or
isolating the combination of the polypeptide or salt substitute thereof and the lipopolysaccharide by dropping the contact product between the polypeptide or salt substitute thereof and the sample in (S1) by gravity using a column, but the present invention is not limited thereto.

In one embodiment of the present invention, the method may comprise isolating or purifying a recombinant protein.

For example, the above-described method may be comprised as one step of the process of isolating and purifying the recombinant protein expressed in gram-negative bacteria from a culture, lysate or extract of gram-negative bacteria transformed with an expression vector including a gene encoding a recombinant protein.

In the culture of the gram-negative bacteria, lipopolysaccharide, as well as the recombinant protein expressed in the gram-negative bacteria, may be comprised, and the lipopolysaccharide comprised in the culture may be removed by the above-described method.

### Advantageous Effects

Since a polypeptide according to the present invention or a salt substitute thereof has high binding ability to lipopolysaccharide, it can have excellent efficiency of removing the lipopolysaccharide.

In addition, in the production of a protein using gram-negative bacteria, the polypeptide according to the present invention or a salt substitute thereof has a high efficiency of removing lipopolysaccharide during purification, so it can maximize the lipopolysaccharide removal efficiency and isolate and purify a large amount of protein with high efficiency.

In addition, the polypeptide according to the present invention or the salt substitute thereof can be reused multiple times, and is economical due to long-term storage stability.

### DESCRIPTION OF DRAWINGS

FIG. 1 illustrates the LPS removal principle of a kit for removing lipopolysaccharide (LPS) according to one embodiment of the present invention.

### MODES OF THE INVENTION

As the present invention may have various modifications and embodiments, specific embodiments of the present invention will be described in further detail below. However, the present invention is not limited to the specific embodiments, and it should be understood that the present invention includes all modifications, equivalents and alternatives included in the technical idea and scope of the present invention. To describe the present invention, when it is determined that a detailed description of the related art may obscure the gist of the present invention, the detailed description thereof will be omitted.

The present invention provides a composition and kit for removing lipopolysaccharide (LPS), which comprises a polypeptide represented by the following sequence general formula or a salt substitute thereof as an active ingredient.

In addition, the present invention provides a use of a polypeptide represented by the following sequence general formula or a salt substitute thereof for removing lipopolysaccharide (LPS):

Ln-X1-L-X2-V-X3-X4-X5-R-X6-L-X7 [General Formula]
in the above general formula,
n is 0 or 1;
L is leucine;
V is valine;
R is arginine;
X1 is lysine (K) or arginine (R);
X2 is glycine (G) or arginine (R);
X3 is glutamic acid (E) or lysine (K);
X4 is alanine (A) or leucine (L);
X5 is lysine (K), arginine (R), or leucine (L);
X6 is tyrosine (Y), alanine (A), tryptophan (W), lysine (K), or aspartic acid (D); and
X7 is aspartic acid (D) or arginine (R),
wherein, however, a polypeptide represented by the sequence of K-L-G-V-E-A-K-R-Y-L-D in the above general formula is excluded.

Hereinafter, the composition and kit for removing lipopolysaccharide (LPS), which comprises a polypeptide according to the present invention or a salt substitute thereof as an active ingredient, and a method of removing lipopolysaccharide will be described in further detail.

Conventionally, the removal of endotoxin from proteins has a big problem that the removal of endotoxin is actually hindered due to the protein's charge state or its intrinsic property such as hydrophobicity, or many products are lost during the removal process. During the mass production of a protein using gram-negative bacteria (e.g., E. coli), a method using chromatography for removing LPS in purification of such a protein was used, but it has many problems in purifying a large amount of protein and also has an economical problem of a price increase in production of a product.

Therefore, the present inventors confirmed through an experiment that the polypeptide according to the present invention or a salt substitute thereof has very high LPS binding ability, and thus it is possible to remove endotoxin with high efficiency, and thus the present invention was completed.

One aspect of the present invention provides a composition and/or kit for removing lipopolysaccharide (LPS), which comprises a polypeptide represented by the following sequence general formula or a salt substitute thereof as an active ingredient.

Ln-X1-L-X2-V-X3-X4-X5-R-X6-L-X7 [General Formula]

In the above general formula,
n is 0 or 1;
L is leucine;
V is valine;
R is arginine;
X1 is lysine (K) or arginine (R);
X2 is glycine (G) or arginine (R);
X3 is glutamic acid (E) or lysine (K);
X4 is alanine (A) or leucine (L);
X5 is lysine (K), arginine (R) or leucine (L);
X6 is tyrosine (Y), alanine (A), tryptophan (W), lysine (K) or aspartic acid (D); and
X7 is aspartic acid (D) or arginine (R),
wherein, however, in the above general formula, a polypeptide represented by the sequence of K-L-G-V-E-A-K-R-Y-L-D is excluded.

In one embodiment of the present invention, the lipopolysaccharide comprises endotoxin.

In one embodiment of the present invention, the polypeptide is any one of 9 types of the polypeptides selected from the group consisting of a) to i):
a) a polypeptide in which, in the above general formula,
n is 0;
X1 is lysine (K);
X2 is glycine (G);
X3 is glutamic acid (E);
X4 is alanine (A);
X5 is lysine (K);
X6 is tyrosine (Y); and
X7 is arginine (R),
corresponding to K-L-G-V-E-A-K-R-Y-L-R, FP3,
b) a polypeptide in which, in the above general formula,
n is 1;
X1 is arginine (R);
X2 is glycine (G);
X3 is glutamic acid (E);
X4 is alanine (A);
X5 is lysine (K);
X6 is tyrosine (Y); and
X7 is arginine (R),
corresponding to L-R-L-G-V-E-A-K-R-Y-L-R, FP5,
c) a polypeptide in which, in the above general formula,
n is 1;
X1 is arginine (R);
X2 is glycine (G);
X3 is glutamic acid (E);
X4 is leucine (L);
X5 is lysine (K);
X6 is tyrosine (Y); and
X7 is arginine (R),
corresponding to L-R-L-G-V-E-L-K-R-Y-L-R, FP6,
d) a polypeptide in which, in the above general formula,
n is 0;
X1 is lysine (K);
X2 is glycine (G);
X3 is glutamic acid (E);
X4 is alanine (A);
X5 is leucine (L);
X6 is tyrosine (Y); and
X7 is aspartic acid (D),
corresponding to K-L-G-V-E-A-L-R-Y-L-D, FP9,
e) a polypeptide in which, in the above general formula,
n is 0;
X1 is arginine (R);
X2 is arginine (R);
X3 is lysine (K);
X4 is leucine (L);
X5 is arginine (R);
X6 is tyrosine (Y); and
X7 is arginine (R),
corresponding to R-L-R-V-K-L-R-R-Y-L-R, FP12,
f) a polypeptide in which, in the above general formula,
n is 0;
X1 is lysine (K);
X2 is arginine (R);
X3 is lysine (K);
X4 is leucine (L);
X5 is arginine (R);
X6 is tyrosine (Y); and
X7 is arginine (R),
corresponding to K-L-R-V-K-L-R-R-Y-L-R, FP13,
g) a polypeptide in which, in the above general formula,
n is 0;
X1 is lysine (K);
X2 is arginine (R);
X3 is lysine (K);
X4 is leucine (L);
X5 is arginine (R);
X6 is alanine (A); and
X7 is arginine (R),
corresponding to K-L-R-V-K-L-R-R-A-L-R, FP13-9a,
h) a polypeptide in which, in the above general formula,
n is 0;
X1 is lysine (K);
X2 is arginine (R);
X3 is lysine (K);
X4 is leucine (L);
X5 is arginine (R);
X6 is tryptophan (W); and
X7 is arginine (R),
corresponding to K-L-R-V-K-L-R-R-W-L-R, FP13-9w, and
i) a polypeptide in which, in the above general formula,
n is 0;
X1 is lysine (K);
X2 is arginine (R);
X3 is lysine (K);
X4 is leucine (L);
X5 is arginine (R);
X6 is lysine (K); and
X7 is arginine (R),
corresponding to K-L-R-V-K-L-R-R-K-L-R, FP13-9k.

The term “polypeptide” used herein refers to a linear molecule formed by bonding of amino acid residues using a peptide bond. The polypeptide of the present invention may be prepared according to a chemical synthesis method known in the art (e.g., solid-phase synthesis techniques) as well as a molecular-biological method (Merrifield, J Amer. Chem. Soc. 85: 2149-54(1963); Stewart, et al., Solid Phase Peptide Synthesis, 2nd. ed., Pierce Chem. Co.: Rockford, 111(1984)).

In addition, the scope of the polypeptide of the present invention may include a biologically functional equivalent having a mutation in an amino acid sequence exhibiting biological activity equivalent to the polypeptide of the present invention. The mutation in an amino acid sequence may be made based on the relative similarity of an amino acid chain substituent, such as hydrophobicity, hydrophilicity, a charge and a size. According to the analysis of the size, shape and type of an amino acid side chain substituent, it can be seen that arginine, lysine and histidine are all positively-charged residues; alanine, glycine and serine have similar sizes; and phenylalanine, tryptophane and tyrosine have similar shapes. Therefore, based on these considerations, arginine, lysine and histidine; alanine, glycine and serine; and phenylalanine, tryptophane and tyrosine may be biologically functional equivalents.

To introduce a mutation, the hydrophobic index of an amino acid may be considered. A hydrophobic index is assigned to each amino acid according to its hydrophobicity and charge. In addition, it is also known that substitutions between amino acids having similar hydrophilicity values result in peptides having equivalent biological activity.

Amino acid exchanges in a peptide that do not overall alter the activity of a molecule are known in the art (H. Neurath, R. L. Hill, The Proteins, Academic Press, New York, 1979). The most commonly-occurring exchanges are the changes between amino acid residues such as Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, or Asp/Gly.

Considering the above-described mutations having biological equivalent activity, the polypeptide of the present invention is interpreted to include a sequence showing substantial identity to the sequence described in the sequence listing. The substantial identity may indicate a sequence having at least 80%, 90% or 95% homology when the sequence of the present invention is aligned to correspond to any other sequence as much as possible, and the aligned sequence is analyzed using an algorithm generally used in the art. Alignment methods for sequence comparison are known in the art (Huang et al., Comp. Appl. BioSci. 8:155-65(1992); Pearson et al., Meth. Mol. Biol. 24:307-31(1994)).

In addition, in one embodiment of the present invention, the polypeptide represented by the sequence general formula according to the present invention may be a peptidomimetic comprising an L-type polypeptide, a D-type polypeptide or a peptoid, or non-natural amino acids.

The D-type polypeptide refers to an allomeric type polypeptide as an enantiomer having the same amino acid sequence as the L-type polypeptide.

In an exemplary embodiment of the present invention, for example, allD FP12-NH₂ (SEQ ID NO: 8) is provided as an allomeric type polypeptide having the same amino acid sequence as FP12-NH₂ (SEQ ID NO: 6).

The term “allD” used herein to designate a polypeptide refers to a D-type polypeptide.

The polypeptide of a peptidomimetic comprising an L-type polypeptide, a D-type polypeptide or a peptoid, or non-natural amino acids may be prepared by various methods known in the art according to a predetermined amino acid sequence.

In one embodiment of the present invention, an end of the polypeptide represented by the sequence general formula according to the present invention may be subjected to alkylation, PEGylation, or amidation.

In an exemplary embodiment of the present invention, among the polypeptides according to the present invention, PEG-allD FP13-NH₂ (AcOH) (SEQ ID NO: 14) is provided by PEGylation of the N-terminus of allD FP13-NH₂ (AcOH) (SEQ ID NO: 13). Although not limited thereto, for PEGylation, the polypeptide according to the present invention may be reacted with Fmoc-NH-PEG2-CH₂COOH, and the molecular weight (Mw) of the polyethylene glycol is approximately 385.4 Da.

The conditions for PEGylation may be adjusted by those of ordinary skill in the art depending on a selected polypeptide, and a PEGylation method may follow various methods known in the art.

The conditions for alkylation or amidation may also be adjusted by those of ordinary skill in the art depending on a selected polypeptide, and the alkylation or amidation method may follow various methods known in the art.

In one embodiment of the present invention, an amine group (NH₂) may be added to the C-terminus of the polypeptide represented by the sequence general formula according to the present invention.

In an exemplary embodiment of the present invention, among the polypeptides according to the present invention, FP12-NH₂ (SEQ ID NO: 6) is provided as a polypeptide in which an amine group (NH₂) is added to the C-terminus of FP12 (SEQ ID NO: 15).

The term “—NH₂” used herein to name a polypeptide refers to an amine group (NH₂) added to the C-terminus of a polypeptide.

The addition of an amine group (NH₂) to the C-terminus of a polypeptide may be performed by various methods known in the art.

In addition, the scope of the polypeptide of the present invention may also include a salt (or salt substitute) thereof.

The salt includes, for example, an acid-addition salt formed by a free acid and a pharmaceutically acceptable metal salt.

Examples of suitable acids may include hydrochloric acid, hydrobromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, gluconic acid, naphthalene-2-sulfonic acid, and benzenesulfonic acid. An acid addition salt may be prepared by a conventional method, for example, by dissolving the compound in an excess of an acidic aqueous solution, and precipitating the salt using a water-miscible organic solvent such as methanol, ethanol, acetone or acetonitrile. In addition, the acid addition salt may be prepared by heating an equimolar amount of a compound and an acid or alcohol in water and then evaporating or drying the mixture, or suction-filtering a precipitate.

Salts derived from suitable bases may include alkali metals such as sodium and potassium, alkaline earth metals such as magnesium, and ammonium, but the present invention is not limited thereto. The alkali metal or alkaline earth metal salt may be obtained, for example, by dissolving a compound in an excess of an alkali metal hydroxide or alkaline earth metal hydroxide solution, filtering an undissolved compound salt, and evaporating and drying the filtrate. Here, as a metal salt, particularly, preparation of a sodium, potassium or calcium salt is pharmaceutically appropriate, and a silver salt corresponding thereto may be obtained by reacting an alkali metal or alkaline earth metal with a suitable silver salt (e.g., silver nitrate).

As part of the salts of the polypeptides described above, the present invention provides acetate salts of the polypeptides described above.

More specifically, an acetate salt substitute of trifluoroacetic acid (TFA) of the polypeptide according to the present invention is provided.

More specifically, the present invention provides an acetate salt of the above-described polypeptide represented by the sequence general formula; a polypeptide such as a peptidomimetic comprising an L-type polypeptide, a D-type polypeptide or a peptoid, or non-natural amino acids; a polypeptide in which its end is subjected to alkylation, PEGylation or amidation; or a polypeptide in which an amine group (NH₂) is added to the C-terminus.

The term “(AcOH)” used herein to designate a polypeptide refers to an acetate salt of a polypeptide.

The polypeptide according to the present invention or a salt substitute thereof has one or more of the following characteristics:
binding ability to lipopolysaccharide (LPS);
being reusable multiple times; or
storage stability, e.g., long-term storage stability.

In an exemplary embodiment of the present invention, the characteristics to be described below of the polypeptide according to the present invention and a salt substitute thereof were confirmed, but the present invention is not limited thereto.

In an exemplary embodiment of the present invention, it was confirmed that the polypeptide according to the present invention and a salt substitute thereof have a strong binding force to the lipopolysaccharide (LPS) (Example 1).

In addition, in an exemplary embodiment of the present invention, it was confirmed that the polypeptide according to the present invention and a salt substitute thereof can be reused multiple times, and preferably, five times or less (Example 4).

In addition, in an exemplary embodiment of the present invention, it was confirmed that the polypeptide according to the present invention and a salt substitute thereof have long-term storage and stability, and thus the LPS removal rate is maintained (Example 7).

Therefore, the polypeptide according to the present invention or a salt substitute thereof; and/or a composition and/or kit comprising the polypeptide or a salt substitute thereof as an active ingredient can be reused multiple times (2 times or more and 10 times or less, 5 times or less, or three times or less).

In addition, the polypeptide according to the present invention or a salt substitute thereof; and/or a composition and/or kit comprising the polypeptide or a salt substitute thereof as an active ingredient has/have storage stability, for example, long-term storage stability.

In one embodiment of the present invention, the polypeptide according to the present invention or a salt substitute thereof may be conjugated to a substrate, and the composition and/or kit according to the present invention may be provided while being conjugated to a substrate.

The substrate may be an insoluble or solid substrate, conjugated with, for example, an agarose bead, for example, sepharose, for example, cyanogen bromide (CNBr)— or N-hydroxysuccinimide (NHS)-binding substrate.

In one embodiment of the present invention, the kit according to the present invention may comprise a container; instructions; and the polypeptide according to the present invention or a salt substitute thereof. The container may serve to package the polypeptide according to the present invention or a salt substitute thereof, and also serve to store and fix it. The material of the container may be, for example, a plastic or glass bottle, but the present invention is not limited thereto. The instructions may describe, for example, the characteristics, configuration or method of using the kit.

In one embodiment of the present invention, the kit may be a spin down type or a column type, but the present invention is not limited thereto.

In the present invention, the spin down-type kit means a kit that can remove lipopolysaccharide by isolating a combination of the polypeptide or a salt substitute thereof and lipopolysaccharide through centrifugation after a lipopolysaccharide-containing sample is added to a tube including the polypeptide according to the present invention or a salt substitute thereof and brought into contact therewith. Here, the polypeptide or salt substitute thereof included in the spin-down type kit may be included in a 1 to 10 mL, 1 to 8 mL, 1 to 6 mL, 3 to 10 mL, 3 to 8 mL, 3 to 6 mL, or 5 mL tube, or separately put into the tube, but the present invention is not limited thereto.

In addition, the column-type kit means a kit that can remove lipopolysaccharide by isolating a combination of a polypeptide according to the present invention or a salt substitute thereof and the lipopolysaccharide after a sample containing the lipopolysaccharide is added to a column including the polypeptide according to the present invention or a salt substitute thereof and brought into contact therewith, the column is mounted on a stand and the sample then drops by gravity without centrifugation. Here, the polypeptide or salt substitute thereof included in the column-type kit may be included in a 1 to 20 mL, 1 to 17 mL, 1 to 15 mL, 1 to 12 mL, 3 to 17 mL, 3 to 15 mL, 3 to 12 mL, 3 to 10 mL, 6 to 10 mL, 6 to 12 mL, 10 to 15 mL, 5 mL, 7.5 mL, or 11.5 mL column, or separately added to the column, but the present invention is not limited thereto.

The present invention provides a method of removing lipopolysaccharide (LPS) from a sample, which comprises the following steps:
(S1) bringing a sample into contact with a polypeptide represented by the above-described sequence general formula, or a salt substitute thereof; and
(S2) isolating the combination of the polypeptide represented by the sequence general formula or a salt substitute thereof and lipopolysaccharide from the sample.

In one embodiment of the present invention, the sample is expected to contain lipopolysaccharide. For example, the sample may be generated in a process of processing gram-negative bacteria.

In one embodiment of the present invention, the method of removing lipopolysaccharide may be comprised in a process of isolating or purifying a recombinant protein.

More specifically, in one embodiment of the present invention, the method of removing lipopolysaccharide may be performed by a part of the process of isolating or purifying a recombinant protein from gram-negative bacteria. Here, for example, the sample comprises a recombinant protein and lipopolysaccharide, and for example, the sample is a culture, lysate or extract of transformed gram-negative bacteria comprising a recombinant protein. For example, the gram-negative bacteria are E. coli.

In one embodiment of the present invention, (S2) may be a step of isolating a combination of the polypeptide or salt substitute thereof and the lipopolysaccharide through centrifugation of the contact product between the polypeptide or salt substitute thereof and the sample in (S1); or
isolating the combination of the polypeptide or salt substitute thereof and the lipopolysaccharide by dropping the contact product between the polypeptide or salt substitute thereof and the sample in (S1) by gravity using a column.

Hereinafter, the present invention will be described in further detail with reference to examples. The examples are merely provided to more specifically explain the present invention, and it will not be construed that the scope of the present invention is limited to the examples according to the gist of the present invention.

### Example 1: Measurement of LPS Binding Ability of Peptide Candidates

FP1 is denoted as wild type (WT), and peptides having point mutations for various parts in WT were prepared (FP3, FP5, FP6, and FP9). To increase the interaction with LPS in the residue sequence of FP3, peptides were designed based on an FP3 and LPS-binding model (FP12-NH₂ and FP13-NH₂), and non-natural amino acid and amino acid isomers (allomeric D-type amino acids) were additionally introduced, thereby designing peptides (allD FP12-NH₂, allD FP13-NH₂, allD FP-13-9a, allD FP-13-9w, allD FP-13-9k, and allD FP13-NH₂ (AcOH)). In addition, an N-terminal PEGylated peptide (PEG-allD FP13-NH₂(AcOH)) was additionally designed and synthesized by Anygen Co. Ltd., and then supplied. Using each of the provided peptides, lipopolysaccharide (LPS), and an isothermal titration calorimeter (ITC) for measuring binding affinity (Kd), binding affinity with LPS was confirmed.

Specifically, a method of analyzing binding affinity (Kd) between a peptide and LPS is as follows.

The binding affinity was measured using Malvern MicroCal PEAQ-ITC cell equipment, and to confirm the interaction with LPS, pre-treatment was performed as follows. The volume and shape of a cell sample were 300 μL, coin-shaped, and fixed-in-place; and the syringe rotation rate was 1,200 rpm; and temperatures were 30° C., 35° C., and 25° C.

Before the test, LPS and the peptide were diluted with PBS in advance, thereby obtaining 2 mM LPS and a 0.2 mM peptide. 300 μL of the peptide was put into cells in an ITC device, which had been washed, and 40 μL of LPS was contained in a syringe. Measurement conditions for ITC (temperature, the number of injections) were set, the syringe was inserted into the cell, and the ITC measurement began. When the measurement was completed, the Kd values of LPS and the peptide were calculated through analysis.

As a result of measurement, for all types of peptides such as FP3, FP5, FP6, FP9, FP12-NH₂, FP13-NH₂, allD FP12-NH₂, allD FP13-NH₂, allD FP-13-9a, allD FP-13-9w, allD FP-13-9k, and allD FP13-NH₂ (AcOH), LPS binding affinity was confirmed, and it was confirmed that they show LPS binding affinity the same as or higher than FP1 (wild type, WT).

TABLE 1

| Sequence | | | Kd |
|---|---|---|---|
| ID | | | (calculated |
| No. | Peptide | Sequence | by ITC) |
| 1 | FP1(WT) | KLGVEAKRYLD | 2.38 × 10⁻⁴ |
| 2 | FP3 | KLGVEAKRYLR | 3.32 × 10⁻⁴ |
| 3 | FP5 | LRLGVEAKRYLR | 8.54 × 10⁻⁴ |
| 4 | FP6 | LRLGVELKRYLR | 1.11 × 10⁻⁵ |
| 5 | FP9 | KLGVEALRYLD | 3.26 × 10⁻⁴ |
| 6 | FP12-NH2 | RLRVKLRRYLR- | 2.42 × 10⁻⁷ |
| | | NH2 | |
| 7 | FP13-NH2 | KLRVKLRRYLR- | 1.18 × 10⁻⁴ |
| | | NH2 | |
| 8 | allD FP12- | RLRVKLRRYLR- | 2.66 × 10⁻⁵ |
| | NH2 | NH2 | |
| | | (all d-form) | |
| 9 | allD FP13- | KLRVKLRRYLR- | 4.78 × 10⁻⁶ |
| | NH2 | NH2 | |
| | | (all d-form) | |
| 10 | allD-FP- | klryklrralr-NH2 | 9.54 × 10⁻⁶ |
| | 13-9a-NH2 | (all d-form) | |
| 11 | allD-FP- | Klrvklrrwlr-NH2 | 1.74 × 10⁻⁵ |
| | 13-9w-NH2 | (all d-form) | |
| 12 | allD-FP- | Klrvklrrklr-NH2 | 1.99 × 10⁻⁶ |
| | 13-9k-NH2 | (all d-form) | |
| 13 | allD-FP-13- | KLRVKLRRYLR- | 1.95 × 10⁻⁵ |
| | NH2 | NH2 | |
| | (AcOH) | (all d-form) | |

TABLE 2

| | | | Kd |
|---|---|---|---|
| Sequence | | | (calculated |
| ID No. | Peptide | Sequence | by ITC) |
| 1 | FP1(WT) | KLGVEAK | 2.38 × 10⁻⁴ |
| | | RYLD | |
| 14 | PEG- | PEG- | 1.75 × 10⁻⁵ |
| | alld FP13- | KLRVKLR | |
| | NH2 (AcOH) | RYLR-NH2 | |
| | | (all d- | |
| | | form) | |
| | | PEG: | |
| | | Fmoc- | |
| | | NH-PEG | |

(In Tables 1 and 2, allD indicates D-type amino acids)

(In Tables 1 and 2, (AcOH) indicates a salt substitute prepared by substituting trifluoroacetic acid (TFA) at a terminus of the 9^{th} amino acid from the N-terminus of the peptide with an acetate salt)

(In Table 2, PEG indicates PEGylation. Information on peptide N-terminal PEGylation: polyethylene glycol (PEG), molecular weight (Mw)=385.4 Da, Fmoc-NH-PEG2-CH₂COOH)

### Example 2: Preparation of Reagent for Removing LPS

According to the method described below, a reagent for removing LPS was prepared by coupling a peptide conjugated to cyanogen bromide (CNBr)-activated agarose.

TABLE 3

| | Component | Molecular | | |
|---|---|---|---|---|
| Classification | Name | Weight | Concentration | |
| Coupling/Wash I | NaHCO₃ | 84.01 | 0.2M | |
| buffer (pH 8.3) | NaCl | 58.44 | 0.5M | |
| Bead solution | HCl | 36.46 | 1 | mM |
| Wash II buffer | NaCl | 58.44 | 0.5M | |
| (pH 4.0) | Sodium acetate | 82.00 | 0.1M | |
| Blocking buffer | Glycine (pH 8.0) | 75.00 | 0.2M | |
| PBS (pH 7.2) | NaCl | 58.44 | 137 | mM |
| | KCl | 74.55 | 2.7 | mM |
| | Na₂HPO₄ | 141.96 | 4.3 | mM |
| | KH₂PO₄ | 136.09 | 1.4 | mM |
| Storage buffer | Glycerol | 92.02 | 50% | |
| | Sodium azide | 65.01 | 0.02% | |
| CNBr | Cyanogen | 105.92 | 25 | mg/mL |
| | Bromide | | | |
| | Activated | | | |
| | Agarose | | | |
| Peptide | 17 types of Adk- | n/a | 2.5 | mg/mL |

| | |
|---|---|
| (example 1) | derived peptide |

### Example 2-1: Mass Production of 300 ml Usage Amount Using Column

(1) Preparation of CNBr-Activated Agarose

7.5 g of cyanogen bromide (CNBr)-activated agarose was measured and reacted (dissolved) in 75 mL of 1 mM HCl for 30 minutes. After inserting a disk into the column, the column was washed with PBS and set (leakage from the column was prevented by disk insertion). After the reaction, the reaction product (dissolution product) was transferred to a column, and washed with 75 mL of 1 mM HCl. 750 mL of distilled water (DW) was added for washing. 375 mL of PBS (pH 7.2) was added for washing.

(2) Polypeptide Binding to CNBr-Activated Agarose

750 mg of a polypeptide was dissolved in 50 mL of coupling/wash I buffer (bead:polypeptide=10:1). The top/bottom cap of the column was closed, and the polypeptide solution was added to the column. The top/bottom cap of the column was sealed and mixing was performed while rotating at 4° C. for 12 to 16 hours.

(3) Washing and Storage

The column was fixed on a stand and the bottom cap was opened to allow the solution to flow out. The bottom cap was closed and 300 mL of 0.2M glycine (pH 8.0) was added and mixed well. The column was fixed on a stand, the bottom cap was opened to allow the solution flow out, and then 300 mL of coupling/wash I buffer was added for washing. 300 mL of wash II buffer was added for washing. The column was fixed on the stand, the bottom cap was opened to allow the solution flow out and a washing process in which 300 mL of coupling/wash I buffer was added and used for washing was repeated four times. A washing process in which 300 mL of PBS (pH 7.2) added and used for washing was repeated twice. 300 mL of a storage solution was mixed with suitable amounts of the completely washed bead/polypeptide and storage buffer. In addition, the resulting product was stored in a 4° C. refrigerator until use.

### Example 2-2: Preparation of Small Usage Amount (1 mL)

25 mg of lyophilized CNBr-activated agarose (hereinafter, “CNBr”) was measured using a micro scale and put into a 5 mL-tube, and then left at room temperature for 30 minutes adding 1 mL of 1 mM HCL thereto. After centrifugation at 1,500 rpm for 1 minute at room temperature, a supernatant was removed using a syringe and CNBr was washed. After adding 1 mL of 1 mM HCl, the tube was mixed up and down and then spun down (1500 rpm, 1 min, room temperature). After removing the supernatant, 10 mL of distilled water was added, and the tube was mixed up and down and spun down (1,500 rpm, 1 min, room temperature). After removing the supernatant, 5 mL of coupling/washing buffer was added, and the tube was mixed up and down and spun down (1,500 rpm, 1 min, room temperature). 1 mL of coupling buffer was added to the beads from which the supernatant was removed. A polypeptide was dissolved to be 2.5 mg/mL and then 1 mL of the polypeptide was added and mixed while rotating at 4° C. for 12 to 16 hours. After centrifuging at 1,500 rpm for 1 minute at room temperature, the supernatant was removed using a syringe, 1 mL of 0.2M glycine (pH 8.0) was added and mixed for 2 hours at room temperature. After centrifugation at 1500 rpm for 1 minute at room temperature, the supernatant was removed (repeated a total of 4 times). After centrifugation by adding coupling buffer by 1 mL (1,500 rpm, 1 min, room temperature), the supernatant was removed, 1 mL of 0.1M acetate and 0.5M NaCl (pH 4.0) were added, followed by centrifugation (1,500 rpm, 1 min, room temperature) (repeated a total of 4 times). After centrifugation under conditions of 1,500 rpm, 1 min and room temperature, the supernatant was removed, and PBS (pH 8.5) was added by 1 mL, followed by centrifugation (1,500 rpm, 1 min, room temperature), the entire process of which was repeated a total of 2 times. The supernatant was removed, 1 mL of PBS (pH 7.4) was added, followed by centrifugation (1,500 rpm, 1 min. RT).

### Example 3-1. Confirmation of LPS Removal Rate of Reagent for Removing LPS

LPS removal rates of the reagent for removing LPS (polypeptide: FP12-NH₂ used) manufactured by the preparation method as described in Example 2 at 10,000 EU/mL and 100,000 EU/mL were checked, and when more than the standard LPS was added to the sample, the degree of LPS removal was confirmed by test repetition.

(1) Test Method for LPS Removal

(1-1) Spin-Down Type

LPS (Sigma, Cat. NO. L4130) was prepared at a concentration of 10,000 EU/mL or 100,000 EU/mL by sonication of 20 mg/mL of a stock aliquoted at a concentration of 500,000 EU/mg or more for 20 minutes and then dilution with PBS. The reagent for removing LPS prepared by the method as described in Example 2-2 was aliquoted at 1 mL into 5 mL tubes. After centrifugation (1,500 rpm, 1 min, room temperature), the supernatant was removed with a syringe, 1 mL of PBS was added and mixed up and down and centrifuged. LPS was dispensed at 10,000 EU/mL and 100,000 EU/mL into tubes, respectively. The resulting product was stirred for 2 hours at room temperature. After centrifugation, the supernatant was transferred to a new tube and used in an LAL test.

(1-2) Column Type

LPS (Sigma, Cat. NO. L4130) was prepared at a concentration of 100,000 EU/mL by sonication of 20 mg/mL of a stock aliquoted at a concentration of 500,000 EU/mg or more for 20 minutes and dilution with PBS. The reagent for removing LPS prepared by the method as described in Example 2-1 was dispensed with an appropriate volume for each column in Column 1 (Thermo 5 mL, cat.no 29922), Column 2 (Rockbourne 7.5 mL, cat.no 104704), and Column 3 (Rockbourne 11.5 mL, cat.no R1010).

After filling each column with a resin, the column was mounted on a stand and washed three times with 5 mL of PBS. When the PBS flowed down, the lower cap was closed, 2 mL of LPS (concentration: 100,000 EU/mL) was added to a sample and then the upper cap was also closed, followed by reacting the sample at room temperature for 2 hours using a rotator which stirs the sample while rotating at 360 degrees. After 2 hours, the column was mounted on a stand, a tube for obtaining a sample was prepared, and after removing the lower cap, the sample falling down the column was received, and the amount of LPS was measured using an LAL kit.

(2) LPS Measurement Method

Reagents and plates used were prepared in advance at room temperature and a heat block was prepared at 37° C. 10 μL of the sample was put into each well of a 96-well plate. 10 μL of an LAL reagent was added. The plate was placed on the bottom, confirmed to be well mixed, and allowed to react at 37° C. for 10 minutes. 20 μL of a substrate was put into each well and shaken on the bottom to allow a reaction at 37° C. for 6 minutes. 20 μL of a stop solution was put into each well and gently shaken, followed by measuring absorbance at 405 nm.

(3) Result

(3-1) Spin-Down Type

Almost 100% of LPS was removed at 10,000 EU/mL and a high removal rate close to 80% was confirmed at 100,000 EU/mL. In addition, in the case of a sample to which LPS was added above the standard (100,000 EU/mL), it was confirmed that 99.9% or more of LPS was removed after repeated removal tests.

TABLE 4

| Feeding LPS | Before | After | Removal | Number of |
|---|---|---|---|---|
| (EU/ml) | (EU) | (EU) | Rate (%) | Removals |
| 10,000 | 10,000 | 10 | 99.9 | 1 |
| 100,000 | 100,000 | 3088 | 79.4 | 1 |
| 100,000 | 100,000 | 100 | 99.9 | 2 |

(3-2) Column Type

As a result of confirming the LPS removal rate under the condition of 100,000 EU/mL of LPS, as shown in Table 5 below, all of Column 1, Column 2, and Column 3 showed an LPS removal effect.

TABLE 5

| | Removal Rate (%) |
|---|---|
| | 100,000 EU |
| Spin-down type | 79.4 |
| Column 1 | 89.88 |
| Column 2 | 75.36 |
| Column 3 | 89.99 |

Therefore, from the above, it was able to be seen that LPS can be removed in two different manners using not only a spin-down type LPS removal method using centrifugation, but also a column type LPS removal method in which a sample flows down by gravity, while mounting on a stand, without centrifugation.

### Example 3-2: Confirmation of Protein Recovery Rates Before and After LPS Removal by Reagent for Removing LPS

To confirm the LPS removal rate of a reagent for removing LPS (polypeptide: FP12-NH₂ used), and whether a protein concentration in a sample is maintained, LPS removability and the protein recovery rate in a sample were confirmed by mixing BSA in the sample.

(1) Test Method for LPS Removal

The reagent for removing LPS was aliquoted at 1 mL into 5 mL tubes. After centrifugation (1,500 rpm, 1 min, room temperature), the supernatant was removed using a syringe, mixed up and down with 1 mL of PBS and then centrifuged. 10,000 EU/mL of LPS and 1 mg/mL of BSA were mixed and then aliquoted. The supernatant was removed using a syringe, and the prepared LPS was aliquoted at 1 mL into tubes. The mixture was stirred for 2 hours at room temperature. After centrifugation, the supernatant was transferred to a new tube, and the LPS removal rate was confirmed through an LAL test. The protein concentration of the supernatant was confirmed by the BCA method below.

(2) Protein Quantification (BCA): Quantitative Analysis of BSA (Protein)

BSA and IgG were quantitatively analyzed by the following method.

Kit: Micro BCA assay (Thermo, Cat. NO. 23235) range: 6.25-100 μg/mL

Standard: The BCA standard (stock: 2 mg/mL) was diluted in DW and 100, 50, 25, 12.5, 6.25, and 0 μg of DW was used as a blank.

As a buffer, 50 mM sodium bicarbonate was prepared.

As a working reagent (WR), 50% solution A+48% solution B+2% solution C (100 μL per well) was prepared.

17 wells (12 standard wells+5 sample wells) were prepared and vortexed for 5 seconds. 100 μL each of the standard (repeated, twice) and the sample were put in a 96 well plate. 100 μL of WR was put in each well and mixed approximately 5 to times by pipetting, and then the 96-well plate was placed on the bottom and shaken up and down. The plate was covered with foil and reacted for 2 hours in a 37° C. incubator, and absorbance was then measured at 562 nm.

(3) Result

When checking the LPS removal rate and protein recovery rate, it was confirmed that the target protein (BSA) in the sample was maintained and only LPS was selectively removed.

TABLE 6

| Sample | LPS Removal Rate (%) | BSA Recovery Rate (%) |
|---|---|---|
| LPS + BSA | 94.8 | 91.8 |

### Example 3-3: Comparison of LPS Removal Rate and BSA Recovery Rate with Other Products when Using the Same Amount of LPS Removal Reagent

The LPS removal rate and protein recovery rate of the reagent for removing lipopolysaccharide (LPS) were confirmed, and the removal efficiency and characteristics were compared using the same amount as those of other commercially available products.

(1) Test Method for LPS Removal

1 mL of a reagent for removing lipopolysaccharide (LPS) (polypeptide: FP12-NH₂ used) and other products (manufactured by B and S companies) with the same amount of bead volume were dispensed into 5 mL tubes. After centrifugation (1,500 rpm, 1 min, room temperature), the supernatant was removed using a syringe and mixed up and down with 1 mL of PBS and then centrifuged. 10,000 EU/mL of LPS and 1 mg/mL of IgG were mixed and aliquoted at 1 mL into tubes. The mixture was stirred for 2 hours at room temperature. After centrifugation, the supernatant was transferred to a new tube, and the LPS removal rate was checked through an LAL test. The protein concentration of the supernatant was determined by the BCA method.

(2) Protein Quantification (BCA): Quantitative Analysis of IgG (Protein)

IgG was quantitatively analyzed by the following method.

Kit: Micro BCA assay (Thermo, Cat. NO. 23235) range: 6.25-100 μg/mL

Standard: The BCA standard (stock: 2 mg/mL) was diluted in DW and 100, 50, 25, 12.5, 6.25, and 0 μg of DW was used as a blank.

As a buffer, 50 mM sodium bicarbonate was prepared.

As a working reagent (WR), 50% solution A+48% solution B+2% solution C (100 μL per well) was prepared.

17 wells (12 standard wells+5 sample wells) were prepared and vortexed for seconds. 100 μL each of the standard (repeated, twice) and the sample were put in a 96 well plate. 100 μL of WR was put in each well and mixed approximately 5 to times by pipetting, and then the 96-well plate was placed on the bottom and shaken up and down. The plate was covered with foil and reacted for 2 hours in a 37° C. incubator, and absorbance was then measured at 562 nm.

(3) Result

The high LPS removal rate of the product of the present invention was confirmed, and it was confirmed that the protein recovery rate was also very high, similar to that of other products.

TABLE 7

| Sample | Feeding LPS | Removal Rate (%) |
|---|---|---|
| Present Invention | 100,000 EU/ml | 79.4 |
| B Company | 100,000 EU/ml | 54.3 |
| S Company | 100,000 EU/ml | 31.5 |

TABLE 8

| Sample | Feeding LPS | IgG Recovery Rate (%) |
|---|---|---|
| Present Invention | 10,000 EU/ml | 97.5 |
| S Company | 10,000 EU/ml | 94.7 |

### Example 4: Confirmation of LPS Removal Rate when Reusing Reagent for Removing LPS

It was confirmed that the removal rate increased when the reagent for removing LPS was used twice to remove 100,000 EU/mL of LPS, and it was confirmed whether the reagent for removing LPS (polypeptide: FP12-NH₂ used) that has been used once can be reused through a regeneration process.

(1) Test Method for LPS Removal

A reagent for removing LPS (polypeptide: FP12-NH₂ used) was aliquoted at 1 mL into 5 mL tubes. After centrifugation, the supernatant was removed using a syringe, 1 mL of PBS was added and mixed up and down and centrifuged. The supernatant was removed using a syringe, and the 100,000 EU/mL of LPS was aliquoted at 1 mL into tubes. The mixture was stirred for 2 hours at room temperature. After centrifugation, the supernatant was transferred to a new tube.

(2) Regeneration

The supernatant of the tube that had been subjected to the LPS removal test was removed, and stirred with 1 mL of 1% sodium deoxycholate (DOC) for 10 minutes at room temperature. After centrifugation, the supernatant was removed with a syringe and washed with 5 mL of 1% DOC. After centrifugation, the supernatant was removed with a syringe and washed with 5 mL of PBS. The resulting mixture was used in an LPS removal ability test.

(3) Result

As shown in Table 9, it was confirmed that the LPS removal rate was maintained at 50% or more until 5 times of use.

TABLE 9

| | | | Removal Rate |
|---|---|---|---|
| Number of | | Removal | Relative to First |
| Reuses | Feeding LPS | Rate (%) | Test (%) |
| Once | 100,000 EU/ml | 60.7 | 100 |
| Twice | 100,000 EU/ml | 64.6 | 106 |
| Three Times | 100,000 EU/ml | 69.6 | 115 |
| Four Times | 100,000 EU/ml | 54.6 | 90 |
| Five Times | 100,000 EU/ml | 52.4 | 86 |

### Example 5: Comparison of LPS Removal Rate by Preparation Batch for Reagent for Removing LPS

A reagent for removing LPS (polypeptide: FP12-NH₂ used) was produced in 200 doses (200 mL) and 300 doses (300 mL) to compare LPS removal rates by product batch.

(1) Test Method for LPS Removal

A reagent for removing LPS was aliquoted by Lot at 2 mL into 5 mL tubes. After centrifugation, the supernatant was removed with a syringe, 2 mL of PBS was added and mixed up and down and centrifuged. After removing the supernatant with a syringe, 100,000 EU/mL of LPS was aliquoted at 1 mL into tubes. The mixture was stirred for 2 hours at room temperature. After centrifugation, the supernatant was transferred to a new tube.

(2) Result

In comparison of the LPS removal rates of two products, as a result of the removal rates for 100,000 EU/mL of LPS, it was confirmed that 96% or more of LPS was removed, showing no significant difference in removal rate.

TABLE 10

| Preparation | | Before | After | Removal |
|---|---|---|---|---|
| Batch | Feeding LPS | (EU) | (EU) | Rate |
| 200rxn | 100,000 EU/ml | 100,000 | 1663 | 98.3 |
| 300rxn | 100,000 EU/ml | 100,000 | 3088 | 96.9 |

### Example 6: Comparison of LPS Removal Rates of Compositions for Removing LPS Using Various Types of Peptides

To confirm an LPS removal rate by preparing a reagent for removing LPS using peptides other than FP12-NH₂, LPS removal rates were compared using the peptides listed in Table 11.

(1) Test Method

Reagents for removing LPS were prepared in one dose by the method of Example 2-2 for each peptide.

More specifically, the reagent for removing LPS was aliquoted by Lot at 2 mL into 5 mL tubes. After centrifugation, the supernatant was removed with a syringe, 2 mL of PBS was added and mixed up and down and centrifuged. The supernatant was removed with a syringe, and LPS 100,000 EU/mL of LPS was aliquoted at 1 mL into tubes. The mixture was stirred for 2 hours at room temperature. After centrifugation, the supernatant was transferred to a new tube.

(2) Result

It was confirmed that all polypeptides exhibited an LPS removal rate of 50% or more. In addition, it was confirmed that, in the case of FP12-NH₂ conjugation, the highest removal rate was exhibited.

TABLE 11

| Peptide | Feeding LPS (EU/ml) | Removal Rate |
|---|---|---|
| FP12-NH2 | 10,000 | 99.9 |
| FP13-NH2 | 10,000 | 94.8 |
| FP12-NH2 | 100,000 | 79.4 |
| allD FP12-NH2 | 100,000 | 58.6 |
| allD FP13-NH2 | 100,000 | 57.7 |

### Example 7: Measurement of LPS Removal Rate—Confirmation of Stability According to Long-Term Storage of Reagent for Removing LPS

To confirm the long-term storage and stability of the composition for removing LPS, LPS removal rates were confirmed for each week.

(1) Test Method

Storage condition for composition for removing LPS: 50% glycerol, 0.02% sodium azide, stored at 4° C.

A test method for LPS removal is as follows.

More specifically, a reagent for removing LPS was aliquoted by Lot at 2 mL into 5 mL tubes. After centrifugation, the supernatant was removed with a syringe, 2 mL of PBS was added and mixed up and down and centrifuged. The supernatant was removed with a syringe, and 100,000 EU/mL of LPS was aliquoted at 1 mL into tubes. The mixture was stirred for 2 hours at room temperature. After centrifugation, the supernatant was transferred to a new tube.

(2) Result

Provided that the LPS removal rate at the 3^{rd} week was 100%, as a result of comparing LPS removal rates, it was confirmed that the LPS removal rate was maintained until 27 weeks. Therefore, the expiration date of the reagent could be set to 6 months or more in storage at 4° C.

TABLE 12

| Week | 3 | 4 | 7 | 8 | 13 | 14 | 20 | 23 | 27 |
|---|---|---|---|---|---|---|---|---|---|
| Removal Rate (%) | 100 | 133.6 | 92.1 | 109.4 | 128.8 | 125.5 | 126.9 | 108.9 | 125.0 |

### Example 8: LPS Removal Principle, Product Composition and Product Specifications of Kit for Removing LPS

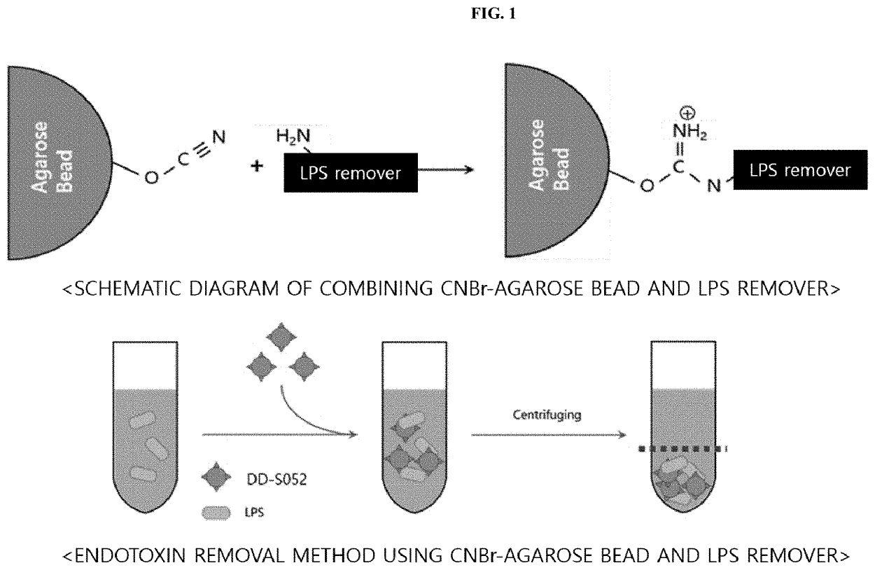

FIG. 1 shows the LPS removal principle of the kit for removing LPS.

In the upper region of FIG. 1, a schematic diagram illustrating the method of coupling an LPS remover (the polypeptide according to the present invention or a salt thereof) to cyanogen bromide (CNBr)-activated agarose (CNBr-agarose beads) is shown.

In addition, in the lower region of FIG. 1, a method of removing an endotoxin using a conjugate of the cyanogen bromide (CNBr)-activated agarose (CNBr-Agarose beads) and the LPS remover (the polypeptide according to the present invention or a salt thereof) (in FIG. 1, referred to as DD-S052) is illustrated.

The product composition of a prototype is shown in Table 13, and product specifications are shown in Table 14.

TABLE 13

| | | | |
|---|---|---|---|
| | Content | Specification | Storage temperature |
| Component | 1 ea | 10 ml (50% glycerol) | (2-8° C.) |

| | |
|---|---|
| Test method | Test method for LPS removal |

TABLE 14

| | |
|---|---|
| Matrix | CNBr agarose bead |
| Volume | 10 ml |
| Min. Binding ability | Binding ability: >4,000,000 EU/ml of bead |
| Storage and Stability | 6 months (2-8° C.) |

As above, as specific parts of the specification have been described in detail, it is clear to those skilled in the art that this specific technique is merely a preferred embodiment, and the scope of the specification is not limited thereto. Thus, the substantial scope of the specification will be defined by the accompanying claims and their equivalents.

### INDUSTRIAL APPLICABILITY

A polypeptide according to the present invention or a salt substitute thereof can be effectively used to maximize lipopolysaccharide removal efficiency and isolate and purify a large amount of protein with high efficiency during purification in the process of producing a protein using gram-negative bacteria.

1. A composition for removing lipopolysaccharide (LPS), comprising:
a polypeptide represented by the following sequence general formula or a salt substitute thereof as an active ingredient:
Ln-X1-L-X2-V-X3-X4-X5-R-X6-L-X7 [General Formula]
in the above general formula,
n is 0 or 1;
L is leucine;
V is valine;
R is arginine;
X1 is lysine (K) or arginine (R);
X2 is glycine (G) or arginine (R);
X3 is glutamic acid (E) or lysine (K);
X4 is alanine (A) or leucine (L);
X5 is lysine (K), arginine (R) or leucine (L);
X6 is tyrosine (Y), alanine (A), tryptophan (W), lysine (K) or aspartic acid (D); and
X7 is aspartic acid (D) or arginine (R),
wherein, however, a polypeptide represented by the sequence of K-L-G-V-E-A-K-R-Y-L-D in the above general formula is excluded.

2. The composition of claim 1, wherein the polypeptide is any one of the 9 types of polypeptides consisting of a) to i) below:
a) a polypeptide in which, in the above general formula,
n is 0;
X1 is lysine (K);
X2 is glycine (G);
X3 is glutamic acid (E);
X4 is alanine (A);
X5 is lysine (K);
X6 is tyrosine (Y); and
X7 is arginine (R),
b) a polypeptide in which, in the above general formula,
n is 1;
X1 is arginine (R);
X2 is glycine (G);
X3 is glutamic acid (E);
X4 is alanine (A);
X5 is lysine (K);
X6 is tyrosine (Y); and
X7 is arginine (R),
c) a polypeptide in which, in the above general formula,
n is 1;
X1 is arginine (R);
X2 is glycine (G);
X3 is glutamic acid (E);
X4 is leucine (L);
X5 is lysine (K);
X6 is tyrosine (Y); and
X7 is arginine (R),
d) a polypeptide in which, in the above general formula,
n is 0;
X1 is lysine (K);
X2 is glycine (G);
X3 is glutamic acid (E);
X4 is alanine (A);
X5 is leucine (L);
X6 is tyrosine (Y); and
X7 is aspartic acid (D),
e) a polypeptide in which, in the above general formula,
n is 0;
X1 is arginine (R);
X2 is arginine (R);
X3 is lysine (K);
X4 is leucine (L);
X5 is arginine (R);
X6 is tyrosine (Y); and
X7 is arginine (R),
f) a polypeptide in which, in the above general formula,
n is 0;
X1 is lysine (K);
X2 is arginine (R);
X3 is lysine (K);
X4 is leucine (L);
X5 is arginine (R);
X6 is tyrosine (Y); and
X7 is arginine (R),
g) a polypeptide in which, in the above general formula,
n is 0;
X1 is lysine (K);
X2 is arginine (R);
X3 is lysine (K);
X4 is leucine (L);
X5 is arginine (R);
X6 is alanine (A); and
X7 is arginine (R),
h) a polypeptide in which, in the above general formula,
n is 0;
X1 is lysine (K);
X2 is arginine (R);
X3 is lysine (K);
X4 is leucine (L);
X5 is arginine (R);
X6 is tryptophan (W); and
X7 is arginine (R), and
i) a polypeptide in which, in the above general formula,
n is 0;
X1 is lysine (K);
X2 is arginine (R);
X3 is lysine (K);
X4 is leucine (L);
X5 is arginine (R);
X6 is lysine (K); and
X7 is arginine (R).

3. The composition of claim 1, wherein the polypeptide is a peptidomimetic comprising an L-type polypeptide, a D-type polypeptide or a peptoid, or non-natural amino acids.

4. The composition of claim 1, wherein an end of the polypeptide is alkylated, PEGylated or amidated.

5. The composition of claim 1, wherein an amine group (NH₂) is added to the C-terminus of the polypeptide.

6. The composition of claim 1, wherein the salt substitute of the polypeptide is an acetate salt substitute.

7. A kit for removing lipopolysaccharide (LPS), comprising:
a polypeptide represented by the following sequence general formula or a salt substitute thereof as an active ingredient:
Ln-X1-L-X2-V-X3-X4-X5-R-X6-L-X7 [General Formula]
in the above general formula,
n is 0 or 1;
L is leucine;
V is valine;
R is arginine;
X1 is lysine (K) or arginine (R);
X2 is glycine (G) or arginine (R);
X3 is glutamic acid (E) or lysine (K);
X4 is alanine (A) or leucine (L);
X5 is lysine (K), arginine (R) or leucine (L);
X6 is tyrosine (Y), alanine (A), tryptophan (W), lysine (K) or aspartic acid (D); and
X7 is aspartic acid (D) or arginine (R),
wherein, however, a polypeptide represented by the sequence of K-L-G-V-E-A-K-R-Y-L-D in the above general formula is excluded.

8. The kit of claim 7, wherein the polypeptide or a salt substitute thereof is conjugated to a substrate.

9. The kit of claim 8, wherein the substrate is a cyanogen bromide (CNBr)-binding agarose bead.

10. The kit of claim 7, wherein the kit is able to be reused multiple times.

11. The kit of claim 7, wherein the kit has long-term storage stability.

12. The kit of claim 7, wherein the kit is a spin-down type or a column type.

13. A method of removing lipopolysaccharide (LPS) from a sample, comprising:
(S1) bringing a sample into contact with a polypeptide represented by the following sequence general formula, or a salt substitute thereof; and
(S2) isolating the combination of the polypeptide represented by the following sequence general formula or a salt substitute thereof and lipopolysaccharide from the sample:
Ln-X1-L-X2-V-X3-X4-X5-R-X6-L-X7 [General Formula]
in the above general formula,
n is 0 or 1;
L is leucine;
V is valine;
R is arginine;
X1 is lysine (K) or arginine (R);
X2 is glycine (G) or arginine (R);
X3 is glutamic acid (E) or lysine (K);
X4 is alanine (A) or leucine (L);
X5 is lysine (K), arginine (R) or leucine (L);
X6 is tyrosine (Y), alanine (A), tryptophan (W), lysine (K) or aspartic acid (D); and
X7 is aspartic acid (D) or arginine (R),
wherein, however, a polypeptide represented by the sequence of K-L-G-V-E-A-K-R-Y-L-D in the above general formula is excluded.

14. The method of claim 13, wherein the sample comprises a recombinant protein and a lipopolysaccharide.

15. The method of claim 13, wherein the (S2) is to isolate a combination of the polypeptide or salt substitute thereof and the lipopolysaccharide through centrifugation of the contact product between the polypeptide or salt substitute thereof and the sample in (S1); or
isolate the combination of the polypeptide or salt substitute thereof and the lipopolysaccharide by dropping the contact product between the polypeptide or salt substitute thereof and the sample in (S1) by gravity using a column.

16. The method of claim 13, wherein the lipopolysaccharide comprises an endotoxin.

17. (canceled)


SEQUENCE LISTING (18 sequences)

SEQ ID NO: 1 - protein, 11 aa, Artificial Sequence
  FP1(WT)
KLGVEAKRYLD

SEQ ID NO: 2 - protein, 11 aa, Artificial Sequence
  FP3
KLGVEAKRYLR

SEQ ID NO: 3 - protein, 12 aa, Artificial Sequence
  FP5
LRLGVEAKRYLR

SEQ ID NO: 4 - protein, 12 aa, Artificial Sequence
  FP6
LRLGVELKRYLR

SEQ ID NO: 5 - protein, 11 aa, Artificial Sequence
  FP9
KLGVEALRYLD

SEQ ID NO: 6 - protein, 11 aa, Artificial Sequence
  FP12-NH2
RLRVKLRRYLR

SEQ ID NO: 7 - protein, 11 aa, Artificial Sequence
  FP13-NH2
KLRVKLRRYLR

SEQ ID NO: 8 - protein, 11 aa, Artificial Sequence
  allD FP12-NH2
RLRVKLRRYLR

SEQ ID NO: 9 - protein, 11 aa, Artificial Sequence
  allD FP13-NH2
KLRVKLRRYLR

SEQ ID NO: 10 - protein, 11 aa, Artificial Sequence
  allD FP-13-9a-NH2
KLRVKLRRALR

SEQ ID NO: 11 - protein, 11 aa, Artificial Sequence
  allD FP-13-9w-NH2
KLRVKLRRWLR

SEQ ID NO: 12 - protein, 11 aa, Artificial Sequence
  allD FP-13-9k-NH2
KLRVKLRRKLR

SEQ ID NO: 13 - protein, 11 aa, Artificial Sequence
  allD FP13-NH2 (AcOH)
KLRVKLRRYLR

SEQ ID NO: 14 - protein, 11 aa, Artificial Sequence
  PEG-allD FP13-NH2 (AcOH)
KLRVKLRRYLR

SEQ ID NO: 15 - protein, 11 aa, Artificial Sequence
  FP12
RLRVKLRRYLR

SEQ ID NO: 16 - protein, 11 aa, Artificial Sequence
  FP13
KLRVKLRRYLR

SEQ ID NO: 17 - protein, 11 aa, Artificial Sequence
  allD FP21-NH2
RLRVKLRRWLR

SEQ ID NO: 18 - protein, 11 aa, Artificial Sequence
  allD FP-13-9d-NH2
KLRVKLRRDLR